(19) United States
(12) Patent Application Publication
(10) Pub. No.: US 2004/0117013 A1
(43) Pub. Date: Jun. 17, 2004

(54) Device and method for treating macular degeneration

(76) Inventor: Ira Schachar, Dallas, TX (US)

Correspondence Address: Docket Clerk, P.O. Drawer 800889, Dallas, TX, 75380, US

(21) Appl. No.: 10/318,340

(22) Filed: Dec. 12, 2002

(51) Int. Cl. A61F 2/16

(52) U.S. Cl. 623/6.36; 623/6.32; 623/6.43; 623/907

(57) ABSTRACT

A device and method is disclosed for treating macular degeneration, an affliction of the eye. An incision is made in the scleral region of the eye and an optical prosthesis for diverging incoming light is inserted and positioned within the vitreous humor proximate to the retina of the eye. The optical prosthesis may be a lens having one or more concave faces. Alternately, the optical prosthesis may be an assembly including a number of optical components. The optical prosthesis intercepts light directed at the macular region of the retina and spreads the light over a larger region of the retina. The optical prosthesis may be anchored or stabilized in a variety of ways prior to closing the incision.

### FIELD OF THE INVENTION

The present invention relates generally to the field of optical prosthetic devices, or implants, and more particularly to an implantable optical device and method for ameliorating the effects of macular degeneration to make it easier for one suffering from the affliction to see.

### BACKGROUND OF THE INVENTION

Advances in medical technology, ever more widely available, along with increasingly safer living environments and healthier lifestyle practices, have led to the increased life expectancy we enjoy today. People, especially those living in developed countries, can expect on average to live lives that are much longer then those of their ancestors of only a few generations ago. Although this phenomenon is generally considered a positive one, it does mean that an ever-larger segment of the population has to deal with the many problems associated with old age. Some of these problems are modest and represent largely an irritation, such as a slow decline of muscular strength and speed, incremental weight gain, the development of wrinkles, and a deterioration of skin tone. Other age-related problems are more significant, however, and some age-related illnesses can be debilitating.

Many, if not most adults also experience a decline in the quality of their eyesight as they age, and often need to resort to reading glasses that they did not need when they were younger. Vision problems usually follow the pattern of other age-related ailments, that is, a slow but steady decline with consequences that can range from mild to severe. Not unexpectedly there is not one, but numerous medical conditions that can lead to a loss of visual acuity, each with their own etiology, or cause.

The present invention is a way to try and counter at least some effects of one such condition, which is known as macular degeneration. Macular degeneration stems, in general, from the destruction of light-sensitive cells in the macula, an area of the retina important for seeing detail. The more cells that are lost, the more severe the effects of the condition. A brief review of the anatomy of the eye, beginning with a description of FIG. 1, will be helpful to more fully understand the phenomenon of macular degeneration, as well as the presently proposed method and device for attempting to lessen its adverse effects.

FIG. 1 is an illustration of the outwardly visible portion of a typical eye 100, in this case a human eye. Behind the transparent cornea 105 can be seen the pupil 110, which appears as a black circle of varying size at the center of the eye, and the iris 115, the colored area surrounding the pupil. The so-called “color” of a person's eye is the color of the pigments contained in the iris, which typically range from blue to dark brown. The “white of the eye” surrounding the iris 115 is the sclera 120. Sclera 120 is a tough, protective layer that helps to maintain the shape of the eye 100. The eyelids 102 above and below eye 100 are not actually part of the eye itself, but rather protective skin flaps that can be extended and retracted to cover and uncover the eye, selectively allowing light to enter and spreading needed moisture on its surface.

FIG. 2 is a cross-sectional view of the eye 100 of FIG. 1 taken along section line A-A. In this view the relationship of the cornea 105 to the pupil 110, iris 115, and sclera 120 is more clearly illustrated. The transparent cornea 105 allows light to enter the eye 100. Light entering through the cornea 105 also passes through the pupil 110, which is not actually a structure but rather an opening formed by the muscles of the iris 115. Iris 115 includes two major sets of muscles (not separately shown or enumerated), namely, the pupillary dilator and the pupillary sphincter for, respectively, dilating and constricting the pupil 110 to control the amount of light entering the eye's interior.

Light rays allowed to pass through the pupil 110 are received at the crystalline lens 125. The lens 125 is generally composed of a set of regularly oriented protein fibers enclosed in a clear capsule. Optically, crystalline lens 125 is a convex lens that focuses the light entering the eye. Lens 125 is sufficiently flexible to allow its shape to be manipulated by the muscles of the ciliary body 130. The muscles of the ciliary body 130 act on the lens 125 through a network of fibers known as zonules 145. The manipulation of the lens 125 allows the eye 100 to focus on objects that are nearer or farther away. The space between the cornea 105 and the lens 125 is divided by the iris into an anterior chamber 135 and a posterior chamber 130, which are filled with a normally clear watery substance known as aqueous humor.

Behind the lens 125 is a main eye chamber 170 filled with vitreous humor, a clear gel-like material. Light entering the eye's interior passes through the vitreous humor on its way to the posterior portion of the eye 100, which contains several important areas that will now be explained.

At the back of the eye 100 lies the retina 150, a seven-layer structure that transduces incoming light rays, converting them from images to neural signals for sending to the brain. Photoreceptors called rods and cones (not shown) are excited when light entering the eye reaches them. A neural signal is transmitted via bipolar cells to ganglion cells (also not shown) that transmit the neural signals created by the rods and cones to the brain. The axons of the ganglion cells from throughout the retina gather at the optic disk 180 and leave the eye 100 through the optic nerve 175.

The photoreceptors are present in greater density in a specialized area called the macula 155, which is located approximately in the center of the retina 150, below and to one side of the optic disk 180. The macula 155 contains in particular a high concentration of cones, the photoreceptors that are sensitive to color, and that are required for sharp, detailed vision. Near the center of the macula 155 is a depression called the fovea 160, where the photoreceptors are exclusively cones.

FIG. 3 is a cross-sectional view of the macular region 155 of the retina 150. Note that for purposes of this illustration the macula 155 in general, and the fovea 160 in particular, are taken to be circular in shape and symmetrical about any section line taken along a diameter, thus no particular section line is delineated in FIG. 2. While this may be substantially if not precisely accurate, it may be taken as true for purposes of this disclosure. In roughly the center of macula 155 the foveal rim 166, a slightly raised (that is, thicker) area of the macula surrounds the foveal slope 164 leading down to the foveal pit 162. The foveal pit 162 is composed of deeply packed cone cells and is, not unexpectedly, the area of sharpest vision of the eye.

The macula 155 generally is therefore a very important region because it allows eye 100 to detect finer detail than the remainder of the retina 150. For this reason, incoming light rays are primarily focused toward the macula 155 by the crystalline lens 125. (The remainder of the retina 150 provides for peripheral vision, motion vision, and night vision.) Note that the common problems of near- and far-sightedness occur when the focal point of the light rays formed by the crystalline lens 125 falls in front of or behind, respectively, the (curved) plane of the retina 150. These are often corrected by external devices such as glasses or contact lenses, which act to readjust the location of the focal point.

Unfortunately, age-related macular degeneration can cause an actual loss of photo-receptors in the important macular area, which reduces the subject's ability to see in a way not easily correctable using external lenses. There may be several contributors to this loss, but the exact etiology that causes some or many of the cone shaped photoreceptors to die is unknown. Although treatments exist that may slow the process of macular degeneration, there is no way to repair the damage that has already occurred. Subjects afflicted with macular degeneration therefore suffer at least some permanent loss of visual acuity.

Adaptive adjustments can be made. If a subject looks to the side of an object they wish to view, they may be able to perceive the object with their peripheral vision, which utilizes unaffected rod and cone cells (the photo-receptors) outside of the macular area. However, the sharpness of the image is decreased. In general this practice takes some getting used to, and can be disconcerting where the object to be viewed is in fact a person. The viewed person may perceive that the macular-degeneration sufferer is not looking at them at all, even though that is precisely what the person with less-than-perfect vision is attempting to do.

It would be desirable for these reasons if a device and method existed that would allow a surgeon to treat macular degeneration using an implanted optical prosthesis that would permit a macular-degeneration sufferer to view objects by “looking” at them more directly and to increase their visual acuity. Accordingly, a need exists in the art for an appropriately constructed device, and a method of implanting it as well. The present invention provides just such a solution.

### SUMMARY OF THE INVENTION

The present invention is directed to a technique for using an intraocular device implanted in the vitreous humor region (main chamber) of the eye, proximate to the retina, to alter the path of incoming light in order to enhance the vision of an eye afflicted with macular degeneration. In one aspect, the present invention is an optical prosthesis such as a lens for implantation in the eye. In one embodiment, the device includes a lens for altering the path of light that would otherwise be directed at (or near) the foveal area of the retina. The lens has two faces, one or both of which may be concave in order to achieve a light-diverging effect. In use the device is placed proximate to the retina near the region of the macula. The device may include a frame to assist in retaining the lens properly in position. The device may also be a compound lens system rather than a single-lens device.

In another aspect, the present invention is a method of treating an eye afflicted with macular degeneration, including the steps of making an incision in the scleral portion of the eye, inserting the optical prosthesis, positioning the optical prosthesis proximate the macular area of the eye, and closing the incision. The method of the present invention may also include the steps of preparing a site within the vitreous humor for placement of the optical prosthesis, and of using a retaining means to retain the optical prosthesis in place.

Accordingly, it is an object of the present invention to provide a treatment for macular degeneration by implanting proximate to the retina of an eye a light-diverging optical device.

It is another object of the present invention to provide a method of treating macular degeneration by using an implant positioned at or near the retina.

Additional objects of the present invention will become apparent from the description of the invention that follows.

The foregoing has outlined rather broadly the features and technical advantages of the present invention so that those skilled in the art may better understand the Detailed Description of the Invention that follows. Additional features and advantages of the invention will be described hereinafter that form the subject matter of the claims of the invention. Those skilled in the art should appreciate that they may readily use the conception and the specific embodiment disclosed as a basis for modifying or designing other structures for carrying out the same purposes of the present invention. Those skilled in the art should also realize that such equivalent constructions do not depart from the spirit and scope of the invention in its broadest form.

Before undertaking the Detailed Description of the Invention, it may be advantageous to set forth definitions of certain words and phrases used throughout this patent document. The terms “include” and “comprise,” and derivatives thereof, mean inclusion without limitation; the term “or” is inclusive, meaning “and/or”; the phrases “associated with” and “associated therewith,” as well as derivatives thereof, may mean to include, be included within, interconnect with, contain, be contained within, connect to or with, couple to or with, be communicable with, cooperate with, interleave, juxtapose, to bound to or with, have, have a property of, or the like; and the term “controller,” “processor,” or “apparatus” means any device, system or part thereof that controls at least one operation. Such a device may be implemented in hardware, firmware or software, or some combination of at least two of the same. It should be noted that the functionality associated with any particular controller may be centralized or distributed, whether locally or remotely. Definitions for certain words and phrases are provided throughout this patent document. Those of ordinary skill should understand that in many instances (if not in most instances), such definitions apply to prior uses, as well as to future uses, of such defined words and phrases.

### BRIEF DESCRIPTION OF THE DRAWINGS

FIG. 1 shows a frontal view of the ordinarily visible portions of an eye, in this case a human eye;

FIG. 2 is a cross-sectional view of the eye of FIG. 1, taken approximately along the section line A-A;

FIG. 3 is a cross-sectional view illustrating specifically the macular region of the eye of FIGS. 1 and 2;

FIG. 4a is a frontal view of an exemplary optical prosthesis according to one embodiment of the present invention; FIG. 4b is a lateral cross-sectional view of the optical prosthesis of FIG. 4a;

FIG. 5a is a frontal view of an exemplary optical prosthesis according to another embodiment of the present invention; FIG. 5b is a lateral cross-sectional view of the optical prosthesis of FIG. 5a;

FIG. 6 is a cross-sectional view of an eye into which the optical prosthesis of FIGS. 4a and 4b has been implanted;

FIG. 7a is a frontal view of an exemplary optical prosthesis according to another embodiment of the present invention; FIG. 7b is a lateral cross-sectional view of the optical prosthesis of FIG. 7a;

FIG. 8 is a cross-sectional view of an eye into which the optical prosthesis of FIGS. 7a and 7b has been implanted;

FIG. 9 is a flow chart illustrating an advantageous embodiment of a method for treating macular degeneration according to an embodiment of the present invention; and

FIG. 10 is a cross-sectional view of an eye into which an optical prosthesis has been implanted and secured according to an embodiment of the present invention.

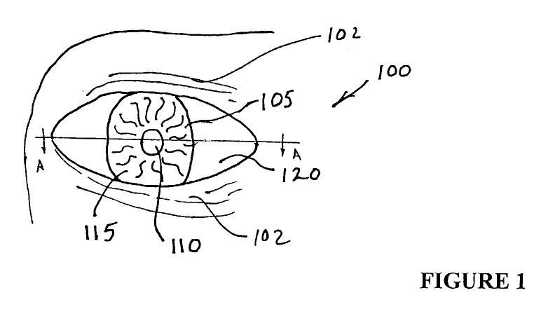

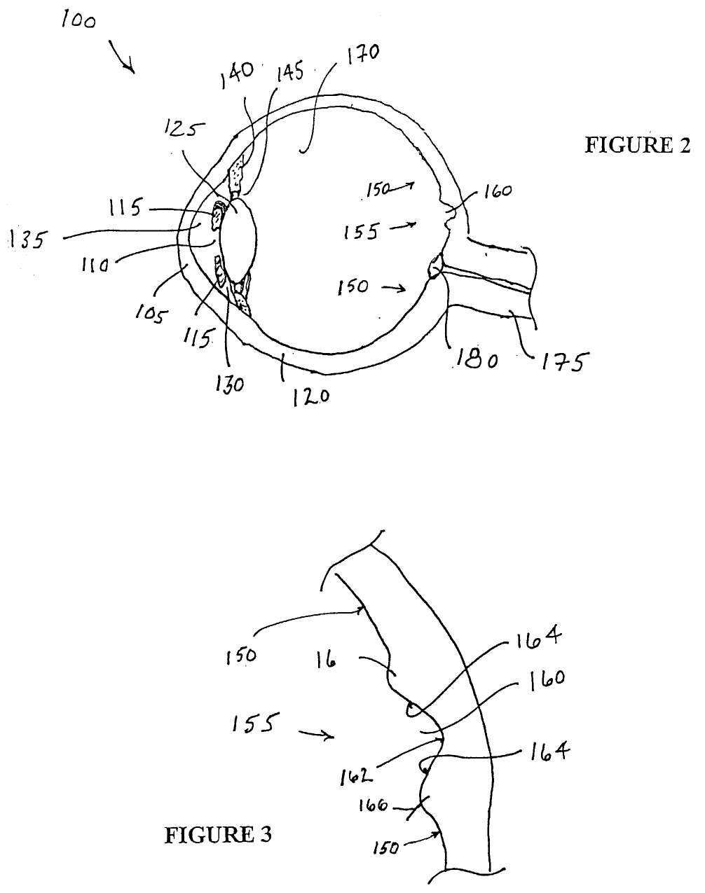

### DETAILED DESCRIPTION OF THE INVENTION

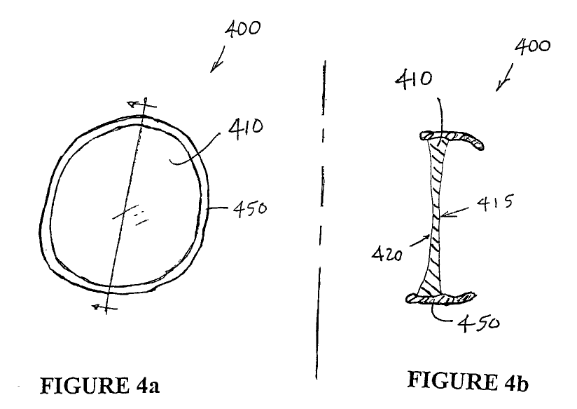

FIGS. 4a through 10, discussed below, and the various embodiments used to describe this principles of the present invention in this patent document are by way of illustration only and should not be construed in any way to limit the scope of the invention. Those skilled in the art will understand that the principles of the present invention may be implemented in any suitably arranged optical device that is implanted proximate to the retina 150. Note that as used herein, “proximate to the retina” refers to a location within the vitreous humor, and preferably closer to the retina than to the crystalline lens. In some applications, the optical prosthesis will touch, or nearly touch the retina, though this contact is not required.

FIGS. 4a and 4b illustrate an optical prosthesis 400 according to an exemplary embodiment of the present invention. In this embodiment, optical prosthesis 400 is a lens 410 surrounded by a frame 450. As in each embodiment, the purpose of the optical prosthesis is to intercept and diverge light as it passes through the optical prosthesis on its way from the crystalline lens 125 of the eye to the retina 150. FIG. 4b is a lateral cross-sectional view of the optical prosthesis 400. In this view it can be seen that the lens 410 has a concave outer surface 420 and concave inner surface 415. A lens so configured will cause light passing through it to diverge, meaning that an image carried by light passing through the lens will impact a larger area of whatever surface it eventually strikes. Note that either the inner surface or the outer surface of lens 410 (or both) may also be convex, or they may be flat (that is, neither convex nor concave). In this application, of course, the surface will be the retina 150. Causing the image to impact a larger retinal area, preferably including portions of the retina 150 outside of the macula 155, is intended to cause the excitation of many additional photoreceptors, including those that have not been destroyed by macular degeneration. Although the resultant perceived image may not be as clear as it would be in an unafflicted (and unaided) eye, it may represent a great improvement over that obtainable without the optical prosthesis.

Lens 410 may be made of, for example, polymethylmethacrylate (PMMA), silicone, polysulfone, glass, or hydrophilic or hydrophobic acrylic materials. Other biocompatible materials may be used as well. In one embodiment, the lens 410 may be a GRIN lens. A GRIN lens is a lens that has a gradient refractive index such that the optical power of the lens varies according to the location at which the light enters the lens. Lens 410 may also be a Fresnel lens. A Fresnel lens is one cut in a series of steps, or formed of a series of prisms more or less concentrically arranged, such that entering light is concentrated and collimated. Similarly, lens 410 may be constructed of one or more prisms for refracting and reflecting the incoming light before it reaches the retina. The type of lens used will, of course, depend on the effect that is sought to be achieved for a particular patient.

In order to further tune the effect produced by the optical device, it or any portion of it may be made of a light-polarizing material. The polarized material may help to control the unnecessary or unwanted dispersion of the light passing through it. The device may also be colored, that is, composed in whole or in part of a material that filters out certain portions of the visible electromagnetic spectrum. In yet another embodiment, the lens 410 is coated with a material for altering, when desired, its optical properties or light-diverging ability. For example, porous silicon or silicon nitrate may be used for this purpose.

The different types of lens materials, treatment, or configuration affect, among other things, its index of refraction. In accordance with various embodiments of the present invention this index may be higher, lower, or equal to the index of refraction of the vitreous humor, the retina, or either or both of them. Where multiple lenses are used, of course, their indices of refraction may vary. Any other material contained by the optical device may, of course, have the same or a different index of refraction.

FIG. 4a is a frontal view of optical prosthesis 400, illustrating that the lens 410 from this perspective is generally circular in shape and entirely surrounded at its peripheral circumference by frame 450. (A circular shape is expected to be desirable in many situations but it is not required.) Frame 450, although optional, may be advantageously employed in some cases. Frame 450 may be integrally formed with the lens 410, or it may be a separately formed component. If separate, it may be attached in a variety of ways, including the use of an adhesive, or mechanical fasteners, or simply sized that it may be press fit.

The frame 450 may have desirable optical characteristics as well, or may be designed to lie, when installed, in an unobtrusive area. The frame 450 may itself in some cases actually interfere somewhat with vision, but the device may be used nevertheless if the overall benefit outweighs this detriment. Note that the frame 450 need not be the same shape as the lens, nor must it be in contact with the lens continuously about an inner circumference (as shown in FIG. 4a and 4b). To serve as a frame, however, it must at some location or locations contact the lens, either directly or through some other intermediate member (not shown). In one alternative embodiment, the intermediate member may include a network of fibers suspending the lens in position. The lens may have separate members attached directly to it without any intermediate frame and, if so, these members may be used to support the lens. Of course, a net (not shown) could be used for anchoring the lens without a frame as well, for example, by fixing the net to the eye 100 in some way or by attaching it to air-fill capsules that would “float” to the top of the vitreous humor and in that way maintain the lens in a relatively (though of course not completely) stable position.

The frame 450, or an attached member, if present, may provide a convenient place to hold the optical prosthesis during insertion and positioning. It may also be used as an aid to proper positioning of the lens in relationship to the retina 150. As light passed through optical prosthesis 400, it is redirected in a diverging fashion, though it will need to pass some distance beyond the inner surface 415 of lens 410 if it is to spread out significantly. In the embodiment of FIGS. 4a and 4b, note how the edge of frame extends beyond the shallowest portion of the concave inner surface 415. When in place, this relationship may provide the desired spacing between the lens 410 and the retina 150. Naturally, the relative inward extension of frame may be changed to match the individual circumstances.

In addition, to the extent that the frame is designed to contact the retina itself, it may also be shaped to increase or reduce the actual contact area, as desired. It may also be formed to an appropriate size to, by its contact with the retina, tend to hold the optical prosthesis in place. Of course, however it is designed, its effect on the subject's overall vision ability should be taken into account.

Where the optical device contacts the retina in such a manner that an enclosed area is created between the device and the retina, lowering the pressure within the enclosed area, relative to the surrounding vitreous humor, may also be used to help anchor the device in place. The pressure differential may be created by evacuating some of the fluid from the enclosed area during or after implantation, or by incorporating into the device a resilient member that can be deformed during implementation in such a way that a pressure differential is created as it attempts to return to its resting shape after removal of the deforming force. A frame comprising a silicone ring, for example, may seal adequately to prevent the differential pressure from equalizing, and may also serve as the resilient member that helps to create the anchoring pressure difference. Alternatively, the optical device is simply a diverging lens that is placed against the retina and is held in position by surface tension.

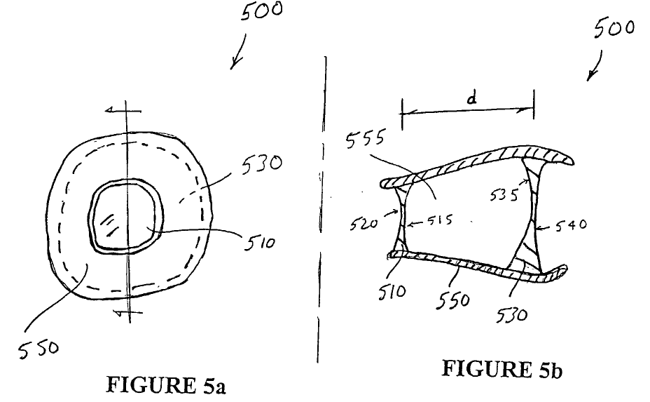

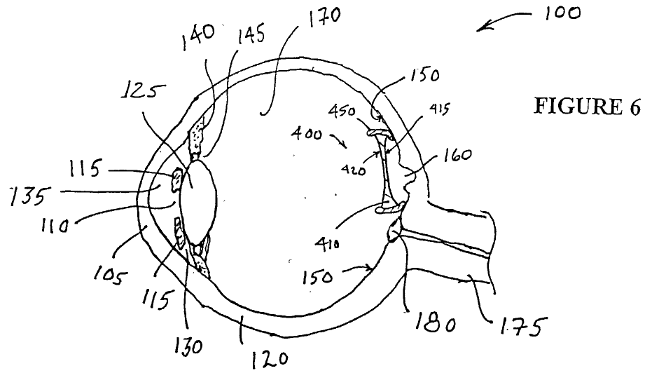

FIGS. 5a and 5b illustrate an optical prosthesis 500 according to another exemplary embodiment of the present invention. FIG. 5a is a frontal view of the device, which in this embodiment includes a first lens 510 and a second lens 530. Prostheses including more than one lens may be referred to as compound-lens prostheses, and the lenses themselves (in combination) as a compound lens system. In this embodiment, first lens 510 has a concave outer surface 520 and a concave inner surface 515. Second lens 530 also has a concave outer surface 540 and a concave inner surface 515, and is separated from first lens 510 by a distance d, a relationship fixed by frame 550. Note that distance d is here shown for clarity to be relatively large, making the optical prosthesis 500 appear somewhat elongate. This configuration is not required, however, and in fact distance d may be quite small. Likewise, first lens 510 is not required to be smaller than second lens 530, as is shown in FIG. 6, although the light passing through first lens 510 will be diverged and a larger second lens 530 may therefore be desirable. Finally, it is not necessary that both lenses be doubly concave in shape.

The cavity 555 formed between the inner surface 515 of first lens 510 and the outer surface 540 of second lens 530 may be filled with air or with some other fluid, depending on the optical qualities desired (or on other considerations). The fluid may, for example, be a viscous fluid such as a gel. In one embodiment the fluid has an index of refraction lower than that of the vitreous humor. In that regard, note that the use of a fluid-filled cavity is not limited to compound lens systems. The lens itself may, for example, be made of a gel having an appropriate refractive index enclosed in a capsule or sac.

In one embodiment (not shown), the gel or other fluid in the cavity may have a refractive index that can be altered by exposure to energy emitted from an energy source, for example, light energy from a laser. Other forms of light energy may be used as well, as may non-light energy from an electrical, magnetic, thermal, or ultra-sound source.

Returning to the embodiment of FIGS. 5a and 5b, cavity 555 may also be provided with one or more openings (not shown) in order to permit the free passage of the surrounding vitreous humor. Naturally, some compensation may have to be made for the volume displaced by optical prosthesis 500, especially of a significant cavity 555 is not allowed to fill. In an alternate embodiment (also not shown), the first lens and the second lens are detachable from each other or from the frame so that they may be separately fitted into place, after which the components may or may not be fixed together. In yet another embodiment (not shown), the first and second lenses may be completely separate components that cooperate even though they are not in physical contact.

FIG. 6 is a cross-sectional view of an eye into which has been implanted the optical prosthesis 400 of FIGS. 4a and 4b. In this embodiment, it can be seen that Frame 450 extends posteriorly to maintain the lens 410 in a spaced relationship with retina 150. In one embodiment, the optical prosthesis 400 is itself large enough to ensure that frame 450 does not significantly impact the retinal area at all, although in some cases retinal contact may be satisfactory. Light rays entering eye 100 through pupil 110 will tend to converge toward a focal point, which in a normally functioning eye would be in the region of the macula 155. As light passes through the lens 410 of optical prosthesis 400, however, the light rays are spread so as to impact retina 150 over a larger area. In this regard, note that the optical prosthesis must be oriented such that this advantageous effect is achieved. This does not mean, however, that any precise orientation is required, though in some cases it may be desirable.

In this way, the wearer of the optical prosthesis 400 may look directly at an object, and still see it to some extent notwithstanding an existing macular degeneration condition. Note that the image perceived by the eye containing optical prosthesis 400 may not perceive the viewed object in the same way that it would have been perceived by a normally functioning eye. Although such a phenomenon would be of great advantage, the object of the present invention is to provide as good and as sharp a view as possible of an object, especially one located directly in front of the eye.

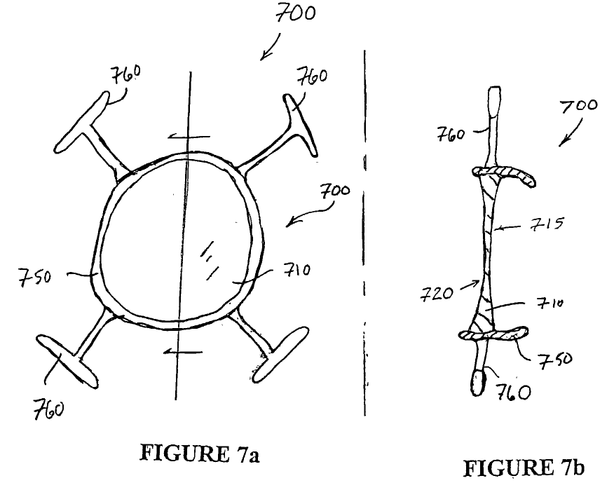

FIGS. 7a and 7b illustrate an optical prosthesis 700 according to another embodiment of the present invention. FIG. 7a is a frontal view of the exemplary optical prosthesis 700, and FIG. 7b is a lateral cross-sectional view. In this embodiment, optical prosthesis 700, which includes lens 710 and frame 750, also includes structural support members 760. The support members may be directly attached to the lens without an intervening frame. The structural members, which are shown here to be symmetrically disposed about the optical prosthesis 700, may be disposed asymmetrically as well. The function of the support members 760 is to contact the inner surface of the eye to assist in holding the optical prosthesis 700 in a fixed orientation, or at a fixed distance from the retina 150, or both.

In this regard, it may also be noted that optical prosthesis 700 may be sized so that its diameter, or alternately that of its lens (or framed lens), is substantially the same size as the macular area of the retina. By “same size” it is meant that the diameter as viewed from the pupil is substantially the same. Note that this dimension will sometimes for convenience be referred to as the “diameter” even if the device (or macula) is not exactly round in shape when viewed from this perspective.

The support members 760 may either be placed against the inner surface of the eye 100, or they may be fastened to it in some way, for example, by using an adhesive substance, a suture sewn into the sclera 120, or even using mechanical fasteners such as tacks that partially or completely penetrate sclera 120. Mechanical fasteners may be made of, for example, titanium, stainless steel, PMMA, or other biocompatible materials. Of course, a combination of fastening means may be used, as illustrated in FIG. 10. FIG. 10 is a cross-sectional view of an eye 100 into which an optical prosthesis 1000 has been implanted according to an embodiment of the present invention. As with embodiments that have been elsewhere described, the optical prostheses 700 and 1000 need not include a frame, although where the supporting members are present, they will preferably attach to the device in some fashion that does not materially affect the desired optical characteristics of the lens 710 (or 1010).

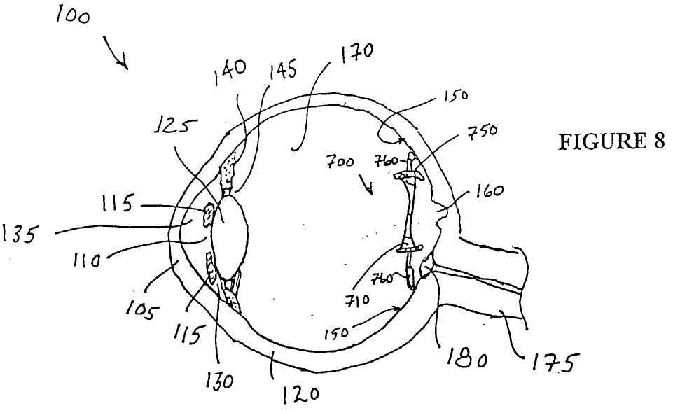

Turning now to the embodiment of FIG. 10, note that as with some of the other embodiments described herein, optical prosthesis 1000 includes a lens 1010, a frame 1050 and supporting members 1060, all of which are in this illustration shown in cross-section. Note that as illustrated here, optical prosthesis 1000 is similar though not identical to the optical device 700 shown, for example, in FIG. 8.

Unlike that device, however, optical prosthesis 1000 is anchored in place using a combination of anchoring means. In this embodiment, a plurality of tacks 1065 are passed through the supporting members 1060 and through the sclera 120, holding optical prosthesis 1000 firmly in position. In addition, suture 1070 wrapped about supporting members 1060 is also passed through the sclera 120 and tied off, providing additional support. Finally, a net 1075 is formed of a plurality of filaments or fibers (not separately enumerated) that also help to hold optical prosthesis 1000 in place. In this embodiment, the fibers of net 1075 are attached to, but do not pass completely through the sclera 120. Note that the embodiment of FIG. 10 is for illustration of various anchoring means, and it is not necessary that all such means are used in combination. Depending on the circumstances, the use of any one or all of them may be desirable. In addition, the use of such anchoring devices does not preclude the circumstance that the optical prosthesis 1000 may also be held in place by other means, such as pressure differential or surface tension. Note also that the use of terms such as “anchoring means”, “anchoring device”, or “held securely (or firmly) in place”, do not indicate that the implanted prosthesis must be completely free of movement in any direction. Such movement may be tolerable, and even in some circumstances desirable. Finally, it is here reiterated that the supporting members, such as supporting members 1060 shown in FIG. 10, as well as the frame 1050, are not required elements for the optical prosthesis. The anchoring means described herein and illustrated, for example, in FIG. 10 may be used whether or not these elements are present. The net 1075, for example, may be particularly advantageous when used with a “lens-only” optical prosthesis (not shown in the illustration).

In yet another alternate embodiment (not shown), the supporting members are not disposed completely around the peripheral of the optical prosthesis, but rest against the eye 100 and help maintain the device's position in certain directions, with, for example, the frame 750 of FIG. 8 also resting against a different portion of the eye and providing additional stability. Note that there may be any number of supporting members.

FIG. 8 is a cross-sectional view of an eye into which the optical prosthesis of FIGS. 7a and 7b has been implanted. As mentioned above, in this embodiment the peripheral portions of the supporting member 760 contact the inner surface of the eye 100, helping to stabilize the optical prosthesis 700 in a fixed relationship with respect to the retina (and specifically, the macula).

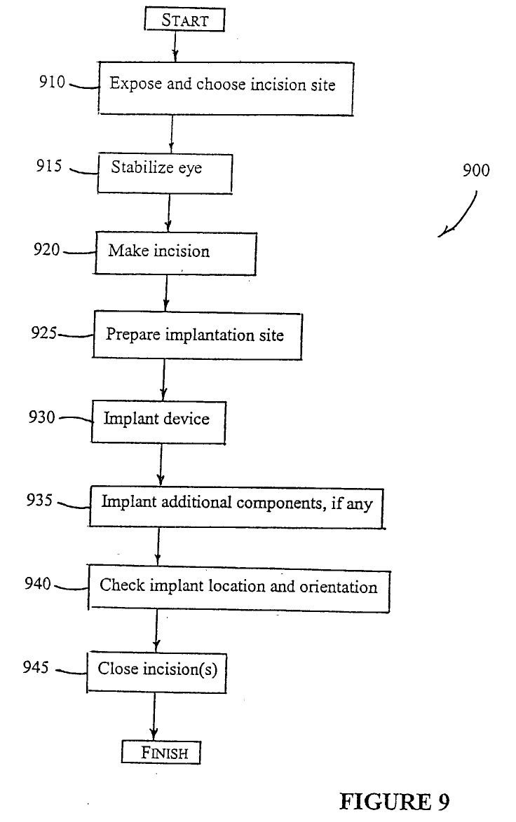

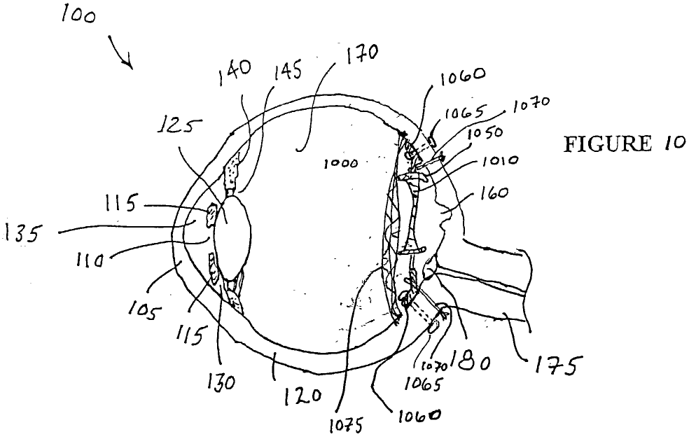

FIG. 9 is a flow chart illustrating a method 900 for treating macular degeneration of an eye according to one embodiment of the present invention. Note that as used herein, a “treatment” is an attempt to correct a known problem by a directed and purposeful action (or forbearance) from which some improvement is reasonable expected. In terms of this disclosure, however, and of the claims reciting the present invention, it is not meant to imply that any level of improvement must actually be realized. In this regard, it is noted that the claims are also intended to cover experimental use of the device and method even if in the experiment itself no improvement in sight is expected. Such would be the case, for example, in cadavers and in animals other than humans. For another example, a person with only one functional eye may agree to the implantation of an optical prosthesis in their non-functional eye in order to test their personal level of acceptance of the device before an attempt is made on the working eye. In these examples, the procedure is performed with no specific expectation of vision improvement, but the claims are nevertheless meant to cover such situations.

Returning to FIG. 9, at Start it is assumed that the patient is prepared for surgery; stabilized and placed in a clean environment and in a convenient orientation, and sedated or anesthetized as necessary under the circumstances. At step 910, a site for making an incision is exposed and chosen, and the eye itself is then stabilized (step 915). An incision of a size appropriate to the optical prosthesis being installed is made (step 920). The site for implantation is then confirmed and probed or prepared as necessary (step 925). The optical prosthesis can then be inserted and properly oriented (step 930). Once in place, any additional optical or structural components may be implanted (step 935), including members for stabilizing the optical prosthesis or attaching it to the eye. The location of the optical prosthesis is then reviewed (step 940) to ensure that it has been properly and securely placed. This may include an ophthalmic examination through the incision itself, through a separate incision created for the purpose, or a visual inspection made through the cornea 105. An optical test (step not shown) may also be conducted at this time.

In an alternate embodiment (also not shown), such an optical test may form an important part of the implantation procedure, with repeated placement and testing of a sequence of optical prostheses to find the one optimum for the eye and the subject being treated. As mentioned above, the optical prosthesis itself may include two or more components that may be detached from one another and interchangeable parts would permit the fitting and testing of a device with varying characteristics. A test procedure is developed to suit the particular eye, perhaps taking into account an assessment of the damaged area, and differently configured optical prostheses are tested to find the one producing the best test result.

Returning to the embodiment of FIG. 9, once proper placement of the optical prosthesis has been confirmed, the incision (or incisions) used for implantation may be closed (step 945). The process may then be repeated in the other eye, if necessary, either in the present procedure or at a future time when the functioning of the first implanted optical prosthesis is confirmed. A separate second procedure may, of course, present the opportunity to make any necessary adjustments to the first installed device. And although repeated opening of each implant-containing eye is not presently considered desirable, it may be necessary to perform the procedure again to install a different optical arrangement if further degradation of the macular area occurs.

The invention having now been fully described, it should be understood that the invention may be embodied in other specific forms or variations without departing from its spirit or essential characteristics. Accordingly, the embodiments described above are to be considered in all respects as illustrative and not restrictive, the scope of the invention being indicated by the appended claims rather than the foregoing description, and all changes which come within the meaning and range of equivalency of the claims are intended to be embraced therein.

What is claimed is:

1. A device for use in an eye afflicted with macular degeneration to improve the ability of the eye to sense notwithstanding the macular degeneration, said device comprising an optical prosthesis for placement within the eye, proximate to a retina of the eye, such that incoming light directed toward a fovea of the eye is diverged so that said incoming light impacts a larger area of the retina.

2. The device as claimed in claim 1, wherein the optical prosthesis comprises a lens.

3. The device as claimed in claim 2, wherein the lens is a gradient refractive index (GRIN) lens.

4. The device as claimed in claim 2, wherein the lens is a Fresnel lens.

5. The device of claim 2, wherein the lens is coated with an optical material.

6. The device of claim 5, wherein the optical material is porous silicon.

7. The device of claim 5, wherein the optical material is silicon nitrate.

8. The device as claimed in claim 2, wherein the lens comprises at least one prism.

9. The device as claimed in claim 2, wherein the lens has an outer surface presented generally in the direction of the pupil of the eye and an inner surface presented generally in the direction of the retina, and wherein at least one of the inner surface and outer surface is flat.

10. The device as claimed in claim 2, wherein the lens has an outer surface presented generally in the direction of the pupil of the eye and an inner surface presented generally in the direction of the retina, and wherein the outer surface is concave.

11. The device as claimed in claim 2, wherein the lens has an outer surface presented generally in the direction of the pupil of the eye and an inner surface presented generally in the direction of the retina, and wherein the inner surface is concave.

12. The device as claimed in claim 2, wherein the lens has an outer surface presented generally in the direction of the pupil of the eye and an inner surface presented generally in the direction of the retina, and wherein the outer surface is convex.

13. The device as claimed in claim 2, wherein the lens has an outer surface presented generally in the direction of the pupil of the eye and an inner surface presented generally in the direction of the retina, and wherein the inner surface is convex.

14. The device as claimed in claim 2, wherein the optical prosthesis comprises a compound lens system.

15. The device as claimed in claim 2, wherein the lens is made of a material from one of: polymethylmethacrylate (PMMA), polysulfone, glass, silicone, and hydrophilic and hydrophobic acrylic material.

16. The device as claimed in claim 2, wherein the lens is substantially similar in diameter to the macula diameter.

17. The device as claimed in claim 2, wherein the lens is substantially larger in diameter than the macular diameter.

18. The device as claimed in claim 2, wherein the lens is retained proximate to the retina by surface tension.

19. The device as claimed in claim 1, wherein the optical prosthesis comprises a fluid-filled cavity that has a lower index of refraction than the vitreous humor or retina.

20. The device as claimed in claim 19, wherein the optical prosthesis comprises a viscous transparent material that has a lower index of refraction than the vitreous humor or retina.

21. The device as claimed in claim 1, wherein the optical prosthesis has a higher index of refraction than the vitreous humor.

22. The device as claimed in claim 1, wherein the optical prosthesis has a higher index of refraction than the retina.

23. The device as claimed in claim 1, wherein the optical prosthesis is made of a light-polarizing material.

24. The device as claimed in claim 1, wherein the optical prosthesis is made of a colored material.

25. A device as claimed in claim 1, wherein the device is held proximate to the retina by biocompatible tacks.

26. The device as claimed in claim 1, wherein the device comprises a material whose index of refraction can be altered by exposure to energy emitted from an energy source.

27. The device as claimed in claim 1, wherein the optical prosthesis is placed immediately adjacent to the retina.

28. The device as claimed in claim 1, further comprising a spacer for maintaining a minimum distance between a plane of light divergence and the retina.

29. The device as claimed in claim 1, further including anchoring means for maintaining the optical prosthesis in a substantially fixed position relative to the retina.

30. The device as claimed in claim 29, wherein the anchoring means is a net.

31. The device as claimed in claim 29, wherein the anchoring means is a pressure differential.

32. The device as claimed in claim 31, wherein the pressure differential is created by evacuating an enclosed area formed between the optical prosthesis and the retina.

33. The device as claimed in claim 31, wherein the device further comprises a resilient member that can be deformed during implantation to create the pressure differential.

34. The device as claimed in claim 33, wherein the resilient member is a silicone ring.

35. A method for treating macular degeneration of an eye, said method comprising the steps of:
providing an optical prosthesis;
making an incision in the sclera of the eye;
inserting the optical prosthesis into the body of the eye through the incision;
placing the inserted optical prosthesis proximate the retina; and
closing the incision.

36. The method of treating macular degeneration as claimed in claim 35, further comprising the step of preparing a site within the eye for placement of the optical prosthesis.

37. The method of treating macular degeneration as claimed in claim 35, wherein the optical prosthesis is a lens.

38. The method of treating macular degeneration as claimed in claim 37, wherein the optical prosthesis has at least one concave surface.

39. The method of treating macular degeneration as claimed in claim 37, wherein the optical prosthesis has at least one convex surface.

40. The method of treating macular degeneration as claimed in claim 37, wherein the optical prosthesis is a compound lens.

41. The method of treating macular degeneration as claimed in claim 37, wherein the optical prosthesis comprises a transparent material that has a lower index of refraction than the vitreous humor or retina.

42. The method of treating macular degeneration as claimed in claim 37, wherein the optical prosthesis comprises a fluid-filled cavity containing a fluid having a lower index of refraction than the vitreous humor or retina.

43. The method of treating macular degeneration as claimed in claim 37, wherein the optical prosthesis comprises a material whose index of refraction can be altered by exposure to energy from an energy source.

44. The method of treating macular degeneration as claimed in claim 37, further comprising the step of securing the optical prosthesis to a sclera of the eye.

45. The method of treating macular degeneration as claimed in claim 37, further comprising the step of securing the optical prosthesis to the retina of the eye.
